(19) United States
(12) Patent Application Publication
(10) Pub. No.: US 2021/0299388 A1
(43) Pub. Date: Sep. 30, 2021

(54) SYSTEMS AND COMPONENTS FOR MULTI-PATIENT MECHANICAL VENTILATION TREATMENT

(71) Applicants: THE REGENTS OF THE UNIVERSITY OF MICHIGAN, Ann Arbor, MI (US); The United States Government as represented by the Department of Veterans Affairs, Washington, DC (US)

(72) Inventors: Kyle VANKOEVERING, Columbus, OH (US); Owen TIEN, Ann Arbor, MI (US); Glenn E GREEN, Gregory, MI (US); David A. ZOPF, Ann Arbor, MI (US)

(21) Appl. No.: 17/219,248

(22) Filed: Mar. 31, 2021

Related U.S. Application Data
(60) Provisional application No. 63/002,506, filed on Mar. 31, 2020.

(51) Int. Cl. A61M 16/08; A61M 16/00; A61M 16/20; A61M 16/06

(52) U.S. Cl. CPC A61M 16/0816; A61M 16/0057; A61M 2205/3331; A61M 16/208; A61M 16/06; A61M 16/0875

(57) ABSTRACT

A system for providing mechanical ventilation to a plurality of patients using a single ventilator is provided. The system includes a first branched adapter may be coupled to a ventilator, the first branched adapter having a plurality of branches and configured to divide a ventilator gas stream into a plurality of gas streams for delivery to a respective plurality of patients. The system includes a pressure regulator in fluid communication with one branch of the first branched adapter and with one patient, the pressure regulator being configured to reduce the pressure of the gas stream reaching the patient such that it is less pressurized than the ventilator gas stream. The system includes a second branched adapter may be coupled to a ventilator, the second branched adapter having a plurality of branches and configured to unite expired gas streams from the plurality of patients into a single expiratory gas stream.

### CROSS-REFERENCE TO RELATED APPLICATIONS

This application claims the benefit of U.S. Provisional Application No. 63/002,506 filed on Mar. 31, 2020. The entire disclosure of the above application is incorporated herein by reference.

### FIELD

The present disclosure relates to a system and associated components for treating a plurality of patients with a single mechanical ventilator.

### BACKGROUND

This section provides background information related to the present disclosure which is not necessarily prior art.

The COVID-19 pandemic has highlighted a troubling reality: in the face of an outbreak causing severe respiratory symptoms, many healthcare facilities around the world do not have enough ventilators to treat all patients requiring ventilation assistance. In the United States, it is expected that the number of patients needing a ventilator will be several times the number of available ventilators. With the rapid escalation in volume of patients requiring support for pulmonary ventilation, mechanical ventilators are quickly becoming a critical limiting resource.

Existing ventilators are designed to oxygenate and ventilate a single patient. While several efforts are underway to manufacture new multi-patient ventilators, a more readily available option is to increase the capacity of existing ventilators intended for use with a single patient to treat instead two or more patients simultaneously. There have been both ex vivo and in vivo tests of ventilator sharing using either a T- or a Y-shape attachment to divide the gas flowing to and from the ventilator. As described, for example, in Neyman, G. et al., “A Single Ventilator for Multiple Simulated Patients to Meet Disaster Surge,” Academic Emergency Medicine 13, 1246-1249 (2006).

These reported efforts have only documented shared ventilator time on the order of hours and are not expected to be feasible for long-term usage. Moreover, with existing techniques for splitting a single ventilator device among multiple patients, pressures and/or flow rates cannot be varied between patients connected to the same ventilator. As a result, a simple splitting adaptor requires multiple patients to have identical lung compliance and ventilatory requirements, or volumes will be preferentially delivered to the more compliant lungs. Risks associated with this lack of differentiation are great. As patients become sicker, lungs become stiffer and therefore require more pressure to adequately fill and function. With ventilator splitting, if ventilator pressure is increased to match the needs of a sicker patient, the healthier patient's lungs may over-inflate, causing serious injury to the lungs. If pressure is reduced to match the needs of the healthier patient, the sicker patient may not receive enough gas to fill the lungs and perform adequate gas exchange. As a result, the sicker patient may experience a dangerous drop in oxygen levels, which can lead to brain injury and death.

In patients with critical respiratory failure, successful split ventilation will ultimately hinge on the capacity to deliver differential ventilation to each patient. Accordingly, whenever ventilator resources are limited, there is a need for components that enable existing ventilator devices to perform differential split ventilation.

### SUMMARY

This section provides a general summary of the disclosure, and is not a comprehensive disclosure of its full scope or all of its features.

The present disclosure is directed to a system for performing differential split ventilation. The system is configured to support a plurality of patients on a single ventilator system.

In certain aspects, the present disclosure relates to a system for providing mechanical ventilation to a plurality of patients using a single ventilator. The system includes: a first branched adapter comprising an inspiratory inlet and a plurality of inspiratory outlets, wherein the inspiratory inlet is directly or indirectly connectable to an inspiratory port of a ventilator; a second branched adapter comprising a plurality of expiratory inlets and an expiratory outlet, wherein the expiratory outlet is directly or indirectly connectable to an expiratory port of a ventilator; and a pressure regulator comprising both a regulator inlet that is directly or indirectly connectable to one of the plurality of inspiratory outlets and a regulator outlet. The pressure regulator is configured to receive a gas stream via the regulator inlet and reduce the pressure of the gas stream before the gas stream exits the regulator outlet.

In certain variations, the present disclosure relates to a system for providing mechanical ventilation to a plurality of patients using a ventilator. The system comprises a first branched adapter comprising a first inlet and a plurality of first outlets fluidly coupled with the first inlet. The first inlet is in fluid communication with an inspiratory port of the ventilator to receive inspiratory fluid therefrom and each of the plurality of first outlets are in fluid communication with a plurality of conduits to each of the plurality of patients and configured to provide inspiratory fluid thereto. The system also comprises a second branched adapter comprising a plurality of second inlets and a second outlet fluidly coupled with the plurality of second inlets. Each of the plurality of second inlets are in fluid communication with a plurality of conduits from each of the plurality of patients and configured to receive expiratory fluid flow therefrom and the second outlet is in fluid communication with an expiratory port of the ventilator. The system also includes a first pressure regulator comprising a regulator inlet that is in fluid communication with at least one of the plurality of first outlets of the first branched adapter and a regulator outlet fluidly coupled to the regulator inlet and in fluid communication with at least one of the plurality of patients. The first pressure regulator is configured to receive the inspiratory fluid via the regulator inlet and reduce a pressure of the inspiratory fluid that exits the regulator outlet.

In certain aspects, the first inlet of the first branched adapter is directly or indirectly connectable to the inspiratory port of the ventilator, the second outlet of the second branched adapter is directly or indirectly connectable to the expiratory port of the ventilator, and the regulator inlet of the first pressure regulator is directly or indirectly coupled to one of the plurality of first outlets of the first branched adapter.

In certain aspects, the first pressure regulator is manually tunable to control an amount of pressure reduction performed.

In certain aspects, the first pressure regulator comprises: a first chamber fluidly coupled to the regulator inlet, a second chamber fluidly coupled to the regulator outlet and the first chamber, a second outlet in fluid communication with the second chamber, an adjustable cap disposed over the second chamber. The adjustable cap is configured to manually adjust a pressure of fluid exiting the second outlet.

In certain aspects, the first pressure regulator comprises: a first chamber fluidly coupled to the regulator inlet, a second chamber fluidly coupled to the regulator outlet and the first chamber, a second outlet in fluid communication with the second chamber, an adjustable cap disposed over the second chamber, a spring disposed beneath the adjustable cap, a piston at least partially disposed within the second chamber. The spring is configured to apply compressive force to the piston via the adjustable cap. A seal component is coupled to the piston so that the piston and the seal component translate from a first operational position of the first pressure regulator, where fluid flow is permitted between the first chamber and the second chamber, to a second operational position where the seal component seals the first chamber from the second chamber to prevent fluid flow.

In certain aspects, at least one of the first pressure regulator, the first branched adapter, and the second branched adapter has a port for fluid communication with a pressure monitor.

In certain aspects, the system further comprises a second pressure regulator, the second pressure regulator comprising a flow regulator component comprising an inlet and an outlet, and a housing encasing at least a portion of the flow regulator component and configured to receive fluid exiting the outlet of the flow regulator component and direct it to an outlet of the housing.

In a further aspect, the system further comprises circuit tubing connectable to the first branched adapter, the first pressure regulator, the second pressure regulator, the second branched adapter, and a patient-interfacing device.

In certain further aspects, the circuit tubing comprises a tube and forms a first conduit through which an inspiratory gas stream can flow from the first branched adapter, through the first pressure regulator, and through the patient-interfacing device to one of the plurality of patients or forms a second conduit through which an expiratory gas stream can flow from one of the plurality of patients, through the patient-interfacing device, optionally through the second pressure regulator, and through the second branched adapter to the ventilator.

In certain aspects, the system further comprises a one-way valve.

In certain aspects, the one-way valve is disposed within a branch of the first branched adapter or second branched adapter or proximal to and in-line with the first branched adapter or the second branched adapter.

In other variations, the present disclosure relates to an adapter assembly for a ventilator system that provides mechanical ventilation to a plurality of patients using a ventilator. The adapter assembly comprises a first branched adapter comprising a first inlet and a plurality of first outlets fluidly coupled with the first inlet. The first branched adapter is configured to be connected to conduits to the plurality of patients and configured to receive inspiratory fluid from the ventilator. The adapter assembly also comprises a second branched adapter comprising a plurality of second inlets and a second outlet fluidly coupled with the plurality of second inlets. The second branched adapter is configured to be connected to conduits from the plurality of patients and configured to receive expiratory fluid flow from the plurality of patients. The adapter assembly also comprises at least one pressure regulator comprising a regulator inlet that is in fluid communication with a regulator outlet fluidly coupled to the regulator inlet, wherein the at least one pressure regulator adjusts or maintains a pressure of at least one of the conduits.

In yet other aspects, the present disclosure relates to a method of providing mechanical ventilation to a plurality of patients using a single ventilator. The method comprises attaching a first branched adapter directly or indirectly to an inspiratory port of a ventilator, the first branched adapter having a plurality of branches and adapted to divide an inspiratory fluid released by the ventilator at a first pressure into a plurality of inspiratory streams for delivery to a plurality of patients. The method comprises attaching a second branched adapter directly or indirectly to an expiratory port of the ventilator, the second branched adapter having a plurality of branches and adapted to unite a plurality of expiratory streams emitted by the plurality of patients into a single flow of expiratory fluid for return to the expiratory port. The method further comprises attaching a pressure regulator directly or indirectly to one of the plurality of branches of the first branched adapter, wherein the pressure regulator is configured to receive a first of the plurality of inspiratory streams at the first pressure and reduce the first pressure to a second pressure. Finally, the method comprises attaching a plurality of conduits to fluidly couple each of the plurality of patients to a respective branch of the first branched adapter and a respective branch of the second branched adapter such that each patient is connected to a respective inspiration line and a respective expiration line, wherein a first patient is connected to a first inspiration line having the pressure regulator disposed along and in fluid communication with the first inspiration line. During operation, the ventilator is configured to release the inspiratory fluid at the first pressure and the first patient receives a stream of fluid at the second pressure.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic drawing of one embodiment of a ventilator system configured to provide customizable mechanical ventilation to a plurality of patients. As depicted in FIG. 1, in accordance with certain aspects of the present disclosure, the system includes a ventilator having an inspiratory line port and an expiratory line port, a first branched adapter fluidly coupled to the inspiratory line port for splitting the flow of gas released from the ventilator, a second branched adapter fluidly coupled to the expiratory line port for uniting separate flows of gas returning to the ventilator, a regulator on the inspiratory line of at least one patient, patient-interfacing devices (e.g., facemask, nasal mask, mouthpiece or intubation tubing), and circuit tubing to fluidly couple each patient to the ventilator and the various components within the ventilator circuit.

FIG. 2 is a top view of one embodiment of a y-adapter, also referred to herein as a 2-branch adapter or simply a branched adapter, prepared in accordance with certain aspects of the present disclosure.

FIG. 3 is a perspective view of the branched adapter embodiment of FIG. 2.

FIG. 4 shows a schematic view of a 3-branch adapter prepared in accordance with certain aspects of the present disclosure. For ease of description, many of the embodiments described herein refer to a system for treating two patients, which includes a 2-branch adapter; however, it will be appreciated by those skilled in the art that every embodiment provided herein is intended and contemplated to treat a plurality of patients, such as 2, 3, 4, or more patients. For example, the ventilator system embodiment of FIG. 1 can be configured to treat three patients by replacing the 2-branch adapters with the 3-branch adapters shown in FIG. 4.

FIG. 5 shows a schematic view of a 4-branch adapter prepared in accordance with certain aspects of the present disclosure. The ventilator system embodiment of FIG. 1 can be configured to treat four patients by replacing the 2-branch adapters with the 4-branch adapters shown in FIG. 5.

FIG. 6 is a perspective view of one embodiment of a first pressure regulator for peak inspiratory pressure (PIP) regulation having a cap removed prepared in accordance with certain aspects of the present disclosure.

FIG. 7 is a front profile view of the first pressure regulator of FIG. 6 with its cap removed.

FIG. 8 is a front profile view of the first pressure regulator of FIG. 6 with its cap partially tightened.

FIG. 9 is a cross-sectional view of the first pressure regulator of FIG. 6 having its cap removed and an internal spring shown.

FIG. 10 is an exploded, pre-assembly view of the first pressure regulator of FIG. 6.

FIGS. 11-12 provide cross-sectional views of one embodiment of a first pressure regulator like in FIG. 6 depicted in operation in accordance with certain aspects of the present disclosure, where a piston moves through various positions thereby opening or closing the valves that allow gas to flow between the chambers. FIG. 11 shows the pressure regulator having the piston in a position that prevents fluid flow between chambers, while FIG. 12 shows the pressure regulator having the piston in a position that facilitates fluid flow between chambers.

FIG. 13 is a top view of the first pressure regulator of FIG. 6.

FIG. 14 is a perspective view of one embodiment of a second pressure regulator for positive end-expiratory pressure (PEEP) regulation prepared in accordance with certain aspects of the present disclosure.

FIG. 15 is a cross-sectional view of a two-part housing component for the second pressure regulator in FIG. 14.

FIG. 16 shows a pressure regulator component for the second pressure regulator like that shown in FIG. 14.

FIG. 17 shows the pressure regulator component of FIG. 16 disposed within a two-part housing of FIG. 15 for collecting any gases released by the pressure regulator component to be directed towards the expiratory port of the ventilator.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” “proximal,” “distal” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

As used herein, the ventilator circuit refers to the combination of the tubing (e.g., circuit tubing) that connects a ventilator to a patient and all the components and devices that are connected in-line with the circuit tubing. In addition to the components described in detail herein, the ventilator circuit may include one or more conventional devices used frequently with mechanical ventilation, for example, one or more filters (e.g., N-95 or N-99 filters), heaters, humidifiers, suction catheters, nebulizers, and/or inhalers.

Example embodiments will now be described more fully with reference to the accompanying drawings.

In various aspects, the present disclosure contemplates a system comprising a plurality of components for mechanically ventilating a plurality of patients using a single ventilator. The multi-patient mechanical ventilation system enables the use of a single ventilator while allowing each patient to receive different pressures, namely different peak inspiratory pressure (PIP) and positive end-expiratory pressure (PEEP). The system enables multiple patients with different lung compliance to receive personalized or customizable pressure support from a single ventilator. The system includes a splitting means that divides a ventilator's single gas flow into a plurality of gas flows intended for a plurality of patients, and a pressure regulation means that provides individualized manual pressure control. A means of independent continual pressure monitoring is also provided. The system may further include a ventilation means.

In various aspects, the ventilation means is a mechanical ventilator or other fluid pump, including for example, an automated Ambu bag, modified anesthesia gas hardware, continuous positive airway pressure machine or other positive pressure device, or a portable oxygen generator.

In some aspects, the system includes the splitting means and pressure regulation means as separate components designed to be easily and removably connected to a mechanical ventilator. Such components make it possible to retrofit an existing single-patient ventilator, when needed, for use in treating more than one patient at a time. The components of some variations are configured to securely couple to and function with any brand and model of mechanical ventilator. In some aspects, a ventilator may be originally fabricated for use with the described system, including the splitting means and pressure regulation means, in order to give clinicians the optionality of using the ventilator to treat one or more patients.

In various aspects, the splitting means is a component that divides fluid flow, referred to herein as a branched adapter. The branched adapter may include two, three, four, or more branches. The branched adapter connects in-line with the ventilation means or ventilator. In various aspects, two branched adapters are provided in the system—one in the inspiratory line of the ventilation circuit and one in the expiratory line of the ventilation circuit. In one variation, the branched adapter may have an inlet fluidly coupled to a plurality of outlets, for example, the branched adapter for use in the inspiratory line of the ventilation circuit. Thus, an inlet of the branched adapter may be in fluid communication with an inspiratory port of the ventilator to receive inspiratory fluid (e.g., pressurized oxygen containing gas) therefrom and each of the plurality of outlets are in fluid communication with conduits to each of the plurality of patients and configured to provide inspiratory fluid thereto. In another variation, the branched adapter may have a plurality of inlets attached to an outlet, for example, the branched adapter for use in the expiratory line of the ventilation circuit. Thus, each of the plurality of inlets are in fluid communication with conduits to each of the plurality of patients and configured to receive expiratory fluid flow therefrom (e.g., expelled gas from the patient) and the outlet is in fluid communication with an expiratory port of the ventilator.

In various aspects, the pressure regulation means is a pressure regulator. The pressure regulator in various aspects includes a regulator inlet, a regulator outlet, and one or more pressure regulating chambers. The regulator inlet, regulator outlet, and one or more pressure regulating chambers are fluidly coupled to one another. In one variation, a first pressure regulator is configured to receive the ventilator's inspiratory or pressurized gas via the regulator inlet and serves to reduce the pressure of the gas before releasing it via the regulator outlet. The pressure regulator may be manually tunable to control the amount of pressure reduction performed by the regulator. The pressure regulator may include any suitable means for reducing the pressure delivered to the patient. In some aspects, a piston system is used. In certain aspects, a system including a piston and a diaphragm may be used to set the pressure within the pressure regulator. In other aspects, a diaphragm system is optionally used.

In another variation, a second pressure regulator is configured to receive the expiratory gas being returned to the ventilator via the regulator inlet and to maintain a pressure of the expiratory fluids at or above a minimum pressure level. The output of the second pressure regulator may be directed to the ventilator's expiratory port. When pressure exceeds the predetermined minimum pressure level (or range of desired pressures), excess fluid may be released via the outlet of the second pressure regulator and captured in an enclosed region, where the released fluid may be returned to the ventilator expiratory port. The second pressure regulator may be manually tunable to control the predetermined pressure level performed by the regulator. The second pressure regulator may include any suitable means for reducing the pressure delivered to the patient. In some aspects, a piston system is used. In other aspects, a combined piston and diaphragm system is used or solely a diaphragm system may be used in other variations.

In one aspect, the first pressure regulator may be a peak inspiratory pressure (PIP) regulator that comprises a first chamber fluidly coupled to the regulator inlet, a second chamber fluidly coupled to the regulator outlet and the first chamber, a piston disposed within the second chamber, and a seal component coupled to the piston, wherein the piston and the seal component translate from a first operational position where fluid flow is permitted between the first chamber and the second chamber to a second operational position where the seal component seals the first chamber from the second chamber to prevent fluid flow. The pressure regulator also includes a top portion disposed over the second chamber having a threaded exterior. The top portion can be coupled to an adjustable cap. The top portion includes a spring. As the adjustable cap (e.g., twist cap) is screwed onto a threaded exterior of the top portion, it increases compressive force on the spring. The further the adjustable cap is screwed onto the top portion, the more compression is applied to the spring. The spring is adjacent to the piston.

The adjustable cap thus compresses the spring and places force on the piston. The piston ultimately elevates against the spring's force when the pressure in the upper chamber overcomes the force of the spring. As the compression of the spring is increased by the adjustable cap, the pressure in the upper chamber required to overcome the spring's force increases. As the piston elevates, it is attached to a separate valve system or seal component that seals the lower chamber, preventing any further flow or pressure buildup in the upper chamber. During operation, the first pressure regulator alternates between the first operational state and the second operational state. Thus, in the second operational state, a maximum pressure in the second chamber is reached and the lower chamber is sealed off. Once the chambers are sealed, fluid does not flow between the lower first chamber and the upper second chamber until the pressure in the upper chamber relaxes with exhalation by permitting the gas to flow through the regulator outlet, at which point the pressure in the system drops and the valve returns to the first operational state. In the first operational state, as the pressure reduces in the upper second chamber as it is evacuated, it reduces the tension on the piston and it reopens the first chamber to the upper second chamber to permit fluid to flow therebetween. Overall, the first pressure regulator allows the input pressure to be reduced with variable manual control by the healthcare provider.

In one further aspect, a second pressure regulator may be a positive end-expiratory pressure (PEEP) regulator. The PEEP regulator may also be a separate component designed to be easily connected to an existing ventilator.

In some aspects, one or more one-way valves are positioned within the proximal branches of the branched adapter or disposed between the branched adapter and the patient, including for example, between the branched adapter and the pressure regulator or between the branched adapter and the PEEP regulator. The one-way valve is configured to only allow gas flow in one direction such that inspiratory flow only travels towards the patient and expiratory flow only travels towards the ventilator.

In some aspects of the disclosure, one or more of the branched adapters, first PIP pressure regulator, second PEEP pressure regulator, and one-way valve are fabricated using additive manufacturing or 3D printing techniques or other manufacturing approaches. The components may be formed from medical-grade polymers or metals. By way of example, certain internal components may be formed from polyphenylsulfone or aluminum. In one variation, components may be machined from medical-grade Radel1 (polyphenylsulfone) and the housing is machined from aluminum, with a silicone (platinum-cured polydimethyl siloxane) gasket seal and stainless-steel spring. Medical grade adhesive may be used to affix components to one another to form assemblies.

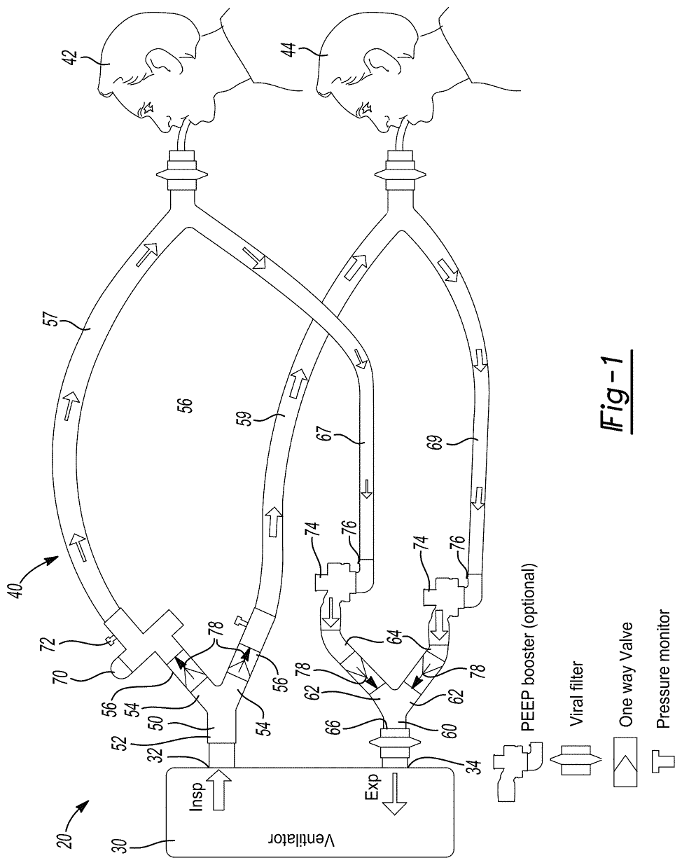

As shown in FIG. 1, a system 20 includes or is used in conjunction with a mechanical ventilator 30 having an inspiratory flow port 32 that delivers a pressurized fluid (e.g., oxygen-containing gas) to a ventilation circuit 40 and an expiratory flow port 34 that receives fluid. The ventilation circuit 40 is in fluid communication with a first patient 42 and a second patient 44. As noted above, the system 20 is not limited to use with only two patients, but is shown with two patients for purposes of illustration. Thus, the expiratory flow port 34 receives fluid exhaled from the first patient 42 and the second patient 44.

The system 20 further includes a first dividing attachment, referred to herein as a first branched adapter 50, in fluid communication with the inspiratory flow port 32. The first branched adapter 50 has a single inlet 52 fluid coupled to a plurality of branches 54 each having a respective outlet 56. The first branched adapter 50 is positioned and configured to split the ventilator's inspiratory gas or fluid flow into a two or more streams of inspiratory fluid flow that flow into conduits, for example, into a first inspiratory flow path 57 directed to the first patient 42 and into a second inspiratory flow path 59 directed to the second patient 44.

The system 20 further includes a second dividing attachment, referred to herein as a second branched adapter 60, in fluid communication with the expiratory flow port 34. The second branched adapter 60 has a plurality of branches 62 each having a respective inlet 64 and a single outlet 66. The second branched adapter 60 is positioned and configured to unite two or more streams of patients' expiratory flow (from the first patient 42 and second patient 44) into a single flow for return to the ventilator 30. The second branched adapter 60 is positioned and configured to join and form the expiratory gas flow returned to the ventilator 30 via the expiratory flow port 34 from two or more streams of expiratory flow or conduits from the patients, for example, from a first expiratory flow path 67 originating from the first patient 42 and from a second expiratory flow path 69 originating from the second patient 44. In various aspects of the disclosure, the first branched adapter 50 and the second branched adapter 60 have an equal number of branches. In various aspects, the number of branches is equal to the number of patients the system 20 is configured to support on a single ventilator 30. For example, a two-branch adapter is configured to support two patients on one ventilator.

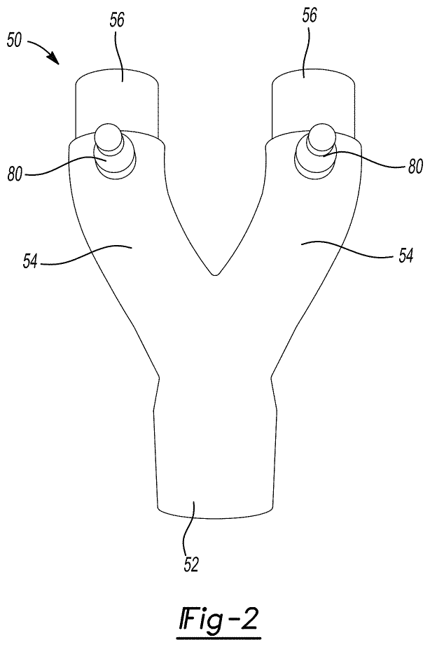

As best seen in FIGS. 2 and 3, a representative first branched adapter 50 is shown that has the single inlet 52 fluid coupled to the plurality of branches 54 each having respective outlets 56. The first branched adapter 50 has the advantage of being an in-line design that does not require significant structural modifications to existing ventilator systems. Thus, the inlet 52 has a female connector that can slide over tubing or a port in the ventilation system. The outlets 56 have a male connector over which a tube or port may be connected. Notably, the connectors may be reversed from the configuration shown, so that the female connectors are male connectors and vice versa, depending on the system requirements. Moreover, while not shown, such connectors may also be used on the second branched adapter 60, which likewise provides an advantageous in-line design. The first branched adapter 50 further includes two optional apertures or ports 80 in each branch 54. These apertures 80 may be standard size IV line ports, so that pressure can be monitored (for example, with an arterial line pressure reducer), or flow rates may be monitored, or therapeutic agents may be introduced into respective branches 54, and the like. When not in use, the apertures 80 can be sealed with a plug or cap.

In the provided view, the ports 80 are standard sized IV line ports that are capped with a universal W line cap. Such caps are removable and the ports are connectable to any device that may be coupled to an IV line port. For example, in various aspects, the ports may be coupled to pressure monitoring devices. In various aspects, the pressure or flow within each branch can be monitored, for example, by connecting a respective arterial line pressure transducer to each port. In various aspects, the ports are also may be coupled to oxygen lines, for example, to deliver more concentrated oxygen to one or more of the patients. While shown in a y-configuration, the 2-branch adapter can take on any shape or configuration suitable for dividing a single flow of fluid into 2 branches of flow. For example, in some embodiments, the adapter may be shaped like a T.

With renewed reference to FIG. 1, the system 20 further includes at least one first pressure regulator 70 positioned in fluid communication with one branch 54 of the first branched adapter 50. In some aspects of the disclosure, only one of the branches 54 of the first branched adapter 50 is in fluid communication with the first pressure regulator 70. In other aspects, two or more of the branches 54 are in fluid communication with one or more first pressure regulators 70 (not shown). In some aspects, while not shown in FIG. 1, each of the branches 54 is in fluid communication with a respective pressure regulator 70. The first pressure regulator 70 provides flexibility in meeting the individual needs of each patient, for example, of first patient 42 and second patient 44. Examples of the first pressure regulator 70 will be described in more detail further below.

In one non-limiting example, with a first pressure regulator 70 present in the system 20, a healthcare professional can increase the pressure of fluid being delivered by the ventilator 30 to match the ventilation needs of the sickest patient (e.g., the sicker of the first patient 42 or the second patient 44 who requires additional oxygen). As noted above, a sicker patient typically has less compliant lungs and may have diminished gas exchange efficiency and therefore requires higher pressures and/or higher levels of oxygenation of inspiratory fluids from a ventilator. By placing the first pressure regulator 70 in fluid communication with the first inspiratory flow path 57 of the healthier first patient 42, the healthcare professional can lower the pressure of the gas emitted from the ventilator 30 to a level suitable to the healthier patient's needs by virtue of the pressure regulator 70.

Stated in another way, the inspiratory fluid flow exiting the inspiratory flow port 32 of the ventilator 30 may be set at a first pressure (P₁) selected for use with the second patient 44 who requires a higher pressure and/or flow rate for assisted pulmonary ventilation. Thus, the inspiratory fluid in the second inspiratory flow path 59 has the first pressure (P₁). However, the pressure regulator 70 adjusts the first pressure by reducing it to a second pressure (P₂) that is tailored to the required pressure and/or flow rate for the first patient's 42 requirements for assisted pulmonary ventilation. Thus, the inspiratory fluid in the first inspiratory flow path 57 delivered to the first patient 42 has a second reduced pressure (P₂), as compared to the first pressure (P₁). In certain aspects, the second reduced pressure (P₂) may be less than or equal to about 7.5 cm water (or about 0.74 kPa), optionally less than or equal to about 8 cm water (or about 0.78 kPa), optionally less than or equal to about 9 cm water (or about 0.88 kPa), optionally less than or equal to about 10 cm water (or about 0.98 kPa), optionally less than or equal to about 15 cm water (or about 1.5 kPa), optionally less than or equal to about 20 cm water (or about 2.0 kPa), optionally less than or equal to about 25 cm water (or about 1,580 kPa), optionally less than or equal to about 25 cm water (or about 2.5 kPa), optionally less than or equal to about 30 cm water (or about 2.94 kPa), optionally less than or equal to about 35 cm water (or about 3.4 kPa), optionally less than or equal to about 40 cm water (or about 3.9 kPa), optionally less than or equal to about 45 cm water (or about 4.4 kPa), and in certain variations, optionally less than or equal to about 50 cm water (or about 4.9 kPa).

The pressure regulator 70 may be adjusted so that it is completely open, so that there is no pressure differential between the patients. The inspiratory fluid flow exiting the inspiratory flow port 32 of the ventilator 30 may be set at a first pressure (P₁) that is delivered to both the first patient 42 and the second patient 44. Where desired, the pressure regulator 70 may be adjusted reduce the first pressure (P₁) to a second pressure (P₂). Thus, in certain non-limiting aspects, a difference in pressure between the first pressure (P₁) and the second pressure (P₂) may be greater than or equal to about 10 cm water (or about 0.98 kPa), optionally greater than or equal to about 15 cm water (or about 1.5 kPa), optionally greater than or equal to about 20 cm water (or about 2.0 kPa), optionally greater than or equal to about 25 cm water (or about 2.5 kPa), optionally greater than or equal to about 30 cm water (or about 2.9 kPa), optionally greater than or equal to about 35 cm water (or about 3.4 kPa), optionally greater than or equal to about 40 cm water (or about 3.9 kPa), optionally greater than or equal to about 45 cm water (or about 4.4 kPa), and in certain variations, optionally greater than or equal to about 50 cm water (or about 4.9 kPa).

The first pressure regulator 70 thereby allows for each patient to receive different peak inspiratory pressures (PIP). This in turn allows for two or more patients with different lung compliance to receive personalized pressure support from one ventilator 30. As shown in FIG. 1, the first pressure regulator 70 also includes an aperture or port 72. The aperture 72 may be standard size IV line ports, so that pressure can be monitored (for example, with an arterial line pressure reducer) and/or flow rates or oxygen levels may be monitored. In other aspects, the aperture 72 may be used to introduce therapeutic agents may be introduced into the first inspiratory flow path 57, and the like or the first pressure regulator 70 may have a plurality of apertures 72.

In some embodiments, only one first pressure regulator is used and it may be placed in the inspiratory line of the healthier patient (e.g., the patient with reduced pressure needs, for example, first pressure regulator 70 disposed in the first inspiratory flow path 57 to the first patient 42). In other embodiments, distinct first pressure regulators (also referred to herein as PIP pressure regulators) may be placed in the inspiratory line of each patient, so that first inspiratory flow path 57 and second inspiratory flow path 59 each have their own PIP pressure regulator (not shown in FIG. 1). Such a configuration allows for accommodation of more than two patients, for example, when 3 or 4 patients are connected to the same ventilator, each with different pressure needs for assisted pulmonary ventilation. Such a setup may also be desired when patients' conditions are variable, for example, when one patient's condition is fluctuating such that at one point in time, the patient is the sicker patient on the ventilator, and at another point in time, the patient is the healthier patient on the ventilator.

The system 20 may further include one or more second pressure regulators 74 (also referred to herein as a PEEP pressure regulator) positioned in fluid communication with the respective inlets 64 of one branch 62 of the second branched adapter 60. In some aspects of the disclosure, only one of the branches 62 of the second branched adapter 60 is in fluid communication with the second pressure regulator 74. In other aspects, two or more of the branches 62 may be in fluid communication with multiple second pressure regulators 74. In some aspects, like that shown in FIG. 1, each of the branches 62 is in fluid communication with a respective second pressure regulator 74. Like the first pressure regulator 70, the second pressure regulator 74 provides flexibility in meeting the individual needs of each patient. Notably, one or more of the first pressure regulator 70 and the second pressure regulator 74 may be omitted from the system 20 depending on patient needs, because in some circumstances the patients may only require different ventilator support on the PIP or inspiratory flow side, while other patients may only require different PEEP support on the expiratory flow side. However, whether the first pressure regulator 70 or the second pressure regulator 74, at least one pressure regulator is advantageously included in the system 20. Examples of the second pressure regulator 74 will be described in more detail further below.

In one non-limiting example, with the second pressure regulator(s) 74 present in the system 20, a healthcare professional can maintain a predetermined pressure of fluid in the first expiratory flow path 67 and/or second expiratory flow path 69. In one example, a sicker patient may require a higher PEEP pressure to prevent less compliant lungs from fully collapsing during exhalation. As noted above, a sicker patient typically has less compliant lungs and therefore may also require a higher pressure in the expiratory fluids returning to a ventilator to minimize collapsing of the lungs. Thus, the pressure in one or more of the expiratory flow paths, for example, in the second expiratory flow path 69 of the second patient 44 may be maintained at a predetermined pressure so that the pressure is higher in the second expiratory flow path as compared to the first expiratory flow path 67. By placing the second pressure regulator 74 in fluid communication with the first and/or second expiratory flow paths 67, 69, the healthcare professional can increase a pressure of the gas in the one of the first and/or second expiratory flow paths 67, 69 while permitting the pressure levels in the other of the first and/or second expiratory flow paths 67, 69 to be lower and tailored to a level suitable to the healthier patient's needs.

The second pressure regulator(s) 74 may have a venting function to vent excess fluids when pressures exceed a predetermined value or range of pressures. It can also be important to measure a volume of gas or fluids exhaled from each patient. Thus, as will be described further below, the second pressure regulator(s) 74 may have an enclosed region that diverts flow of gas having an excess pressure that is returned to the expiratory flow port 34 of the ventilator so that it may be measured.

In certain variations, the expiratory fluid flow of the patient requiring a higher pressure may be set to a third pressure (P₃) selected for use with the second patient 44 who requires a higher PEEP pressure for assisted pulmonary ventilation. Thus, the expiratory fluid in the second expiratory flow path 69 from the second patient 44 may be third pressure (P₃) of greater than or equal to about 5 cm water (or about 316 kPa), optionally greater than or equal to about 10 cm water (or about 632 kPa), optionally greater than or equal to about 15 cm water (or about 948 kPa), optionally greater than or equal to about 20 cm water (or about 1,264 kPa), optionally greater than or equal to about 25 cm water (or about 1,580 kPa), and in certain variations, optionally greater than or equal to about 30 cm water (or about 1,896 kPa).

The second pressure regulator 74 thereby allows for each patient to receive a different positive end-expiratory pressure (PEEP). This in turn allows for two or more patients with different lung compliance to receive personalized pressure support from one ventilator 30. As shown in FIG. 1, the second pressure regulator(s) 74 also include an aperture or port 76. The aperture 76 may be standard size W line ports, so that pressure can be monitored (for example, with an arterial line pressure reducer) and/or flow rates or oxygen levels may be monitored.

In some embodiments, only one pressure regulator is used and it may be placed in the inspiratory line of the healthier patient (e.g., the patient with reduced PIP pressure needs) or the expiratory line of the sicker patient (e.g., the patient with increased PEEP pressure needs). In certain embodiments, distinct pressure regulators may be placed in the expiratory line of each patient, so that first expiratory flow path 67 and second expiratory flow path 69 each have their own pressure regulator (shown in FIG. 1). As discussed above, such configurations allow for accommodation of more than two patients and/or may also be desired when patients' conditions are variable, for example, when one patient's condition is fluctuating such that at one point in time, the patient is the sicker patient on the ventilator, and at another point in time, the patient is the healthier patient on the ventilator.

The system 20 also includes a plurality of one-way valves 78 disposed in the system. The one-way valves 78 are shown disposed in the ventilation circuit 40: (i) between the first branched adapter 50 and the first pressure regulator 70 in the first inspiratory flow path 57 of the first patient 42 (oriented to permit flow from the ventilator 30 towards the first patient 42), (ii) between the first patient 42 and the second branched adapter 60 in the first expiratory flow path 67 (oriented to permit flow from the first patient 42 towards the ventilator 30), (iii) between the first branched adapter 50 and the second patient 44 in the second inspiratory flow path 59 (oriented to permit flow from the ventilator 30 towards the second patient 44), and (iv) between the second pressure regulator 74 and the second branched adapter 60 in the second expiratory flow path 69 (oriented to permit flow from the second patient 44 towards the ventilator 30). The one-way check valves 78 permit flow in the desired direction, but prevent backflow into undesired flow paths within the ventilation circuit 40. Notably, the one-way check valves 78 may be disposed in other locations within the first inspiratory flow path 57, the second inspiratory flow path 59, the first expiratory flow path 67, and the second expiratory flow path 69.

The pressure regulator 70 is manually adjustable allowing healthcare professionals to manage gas flow and pressure to an individual connected to a shared ventilator. Generally, a sufficient fluid flow is maintained when regulating pressure in the pressure regulator 70 without need for additional regulation of fluid flow.

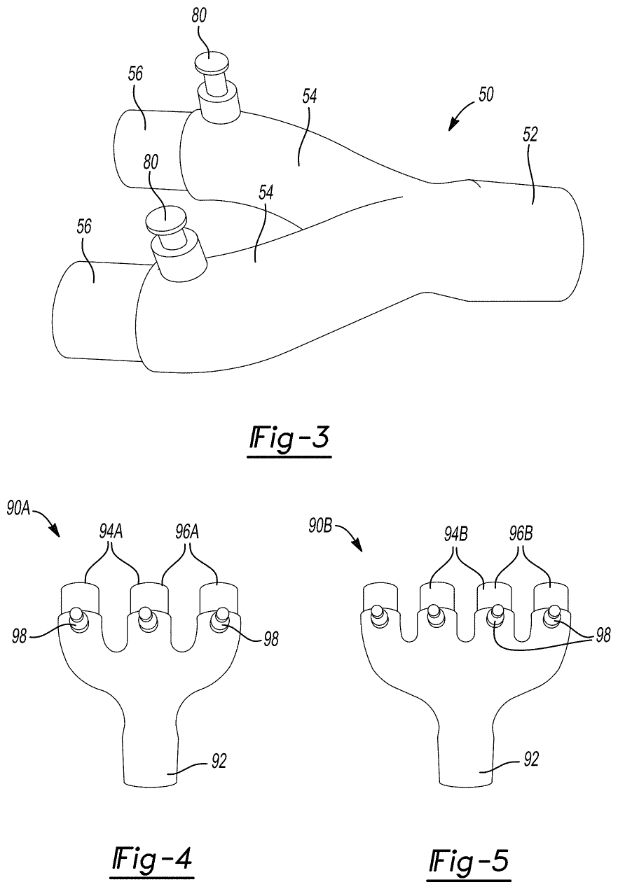

FIGS. 4 and 5 show modified adapters according to certain aspects of the present invention that may be used as either the first branched adapter or the second branched adapter shown in FIG. 1. FIG. 4 shows a 3-branch adapter 90A that can be used in place of the 2-branch adapters (first branch adapter 50 and second branch adapter 60) shown in FIG. 1 to treat three patients. 3-branch adapter 90A has a single inlet 92 fluidly coupled to three branches 94A each having respective outlets 96A. The 3-branch adapter 90A further includes two optional apertures or ports 98 in each branch 94A.

FIG. 5 shows a 4-branch adapter 90B that can be used in place of the 2-branch adapters (first branch adapter 50 and second branch adapter 60) shown in FIG. 1 to treat four patients. 4-branch adapter 90B has a single inlet 92 fluidly coupled to three branches 94B each having respective outlets 96B. The 4-branch adapter 90B further includes two optional apertures or ports 98 in each branch 94B.

The present disclosure also provides methods of providing mechanical ventilation to a plurality of patients using a single ventilator. In one variation, the method comprises attaching a first branched adapter directly or indirectly to an inspiratory port of a ventilator. The first branched adapter has a plurality of branches and is adapted to divide an inspiratory fluid released by the ventilator at a first pressure into a plurality of inspiratory streams for delivery to a plurality of patients. The method also includes attaching a second branched adapter directly or indirectly to an expiratory port of the ventilator. The second branched adapter has a plurality of branches and is adapted to unite a plurality of expiratory streams emitted by the plurality of patients into a single flow of expiratory fluid for return to the expiratory port. A pressure regulator is attached directly or indirectly to one of the plurality of branches of the first branched adapter. The pressure regulator is configured to receive a first of the plurality of inspiratory streams at the first pressure and reduce the first pressure to a second pressure.

The method also includes attaching a plurality of conduits to fluidly couple each of the plurality of patients to a respective branch of the first branched adapter and a respective branch of the second branched adapter. In this manner, each patient is connected to a respective inspiration line and a respective expiration line. A first patient of the plurality of patients is connected to a first inspiration line having the pressure regulator disposed along and in fluid communication with the first inspiration line. When the ventilator is operated, the ventilator releases the inspiratory fluid at the first pressure, and the first patient receives a stream of fluid at the second pressure.

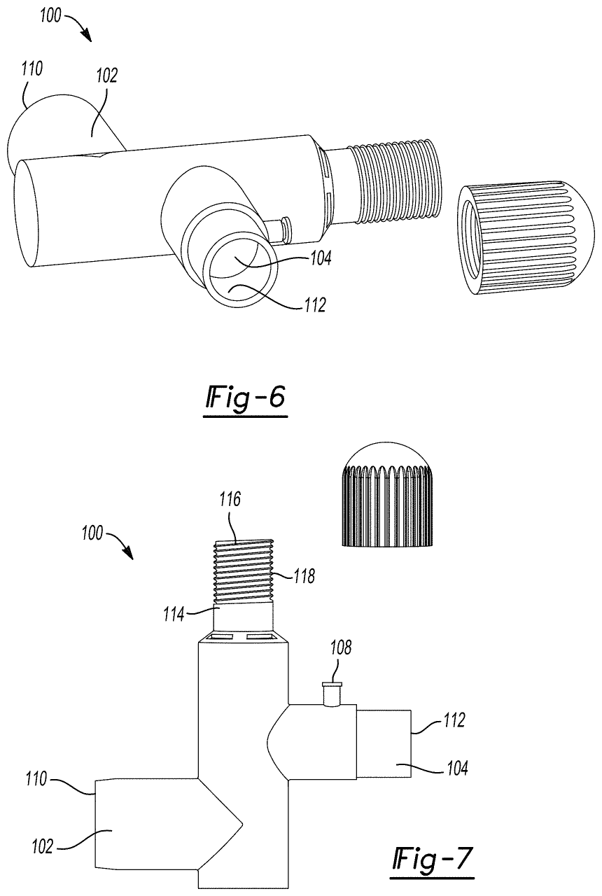

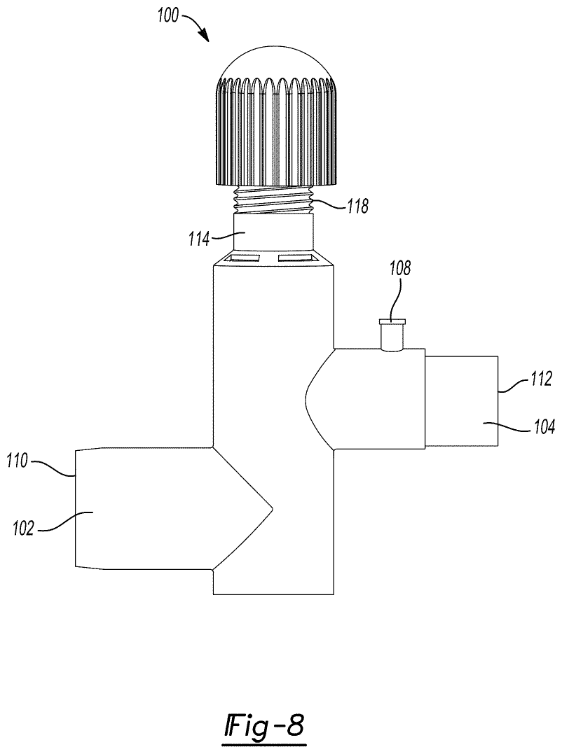

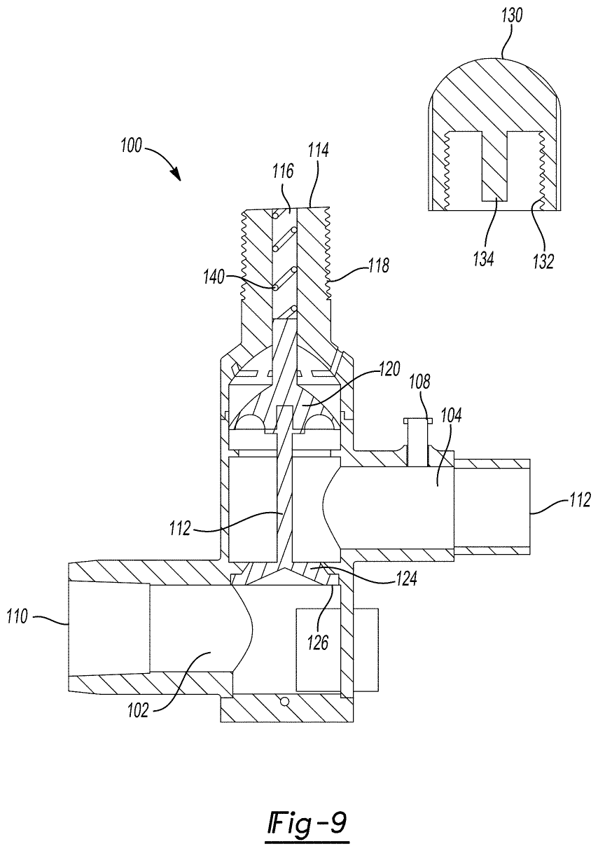

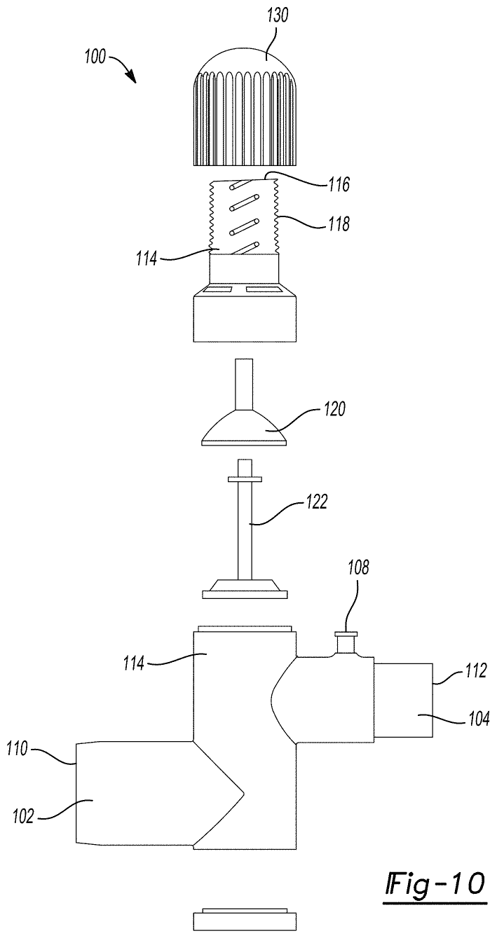

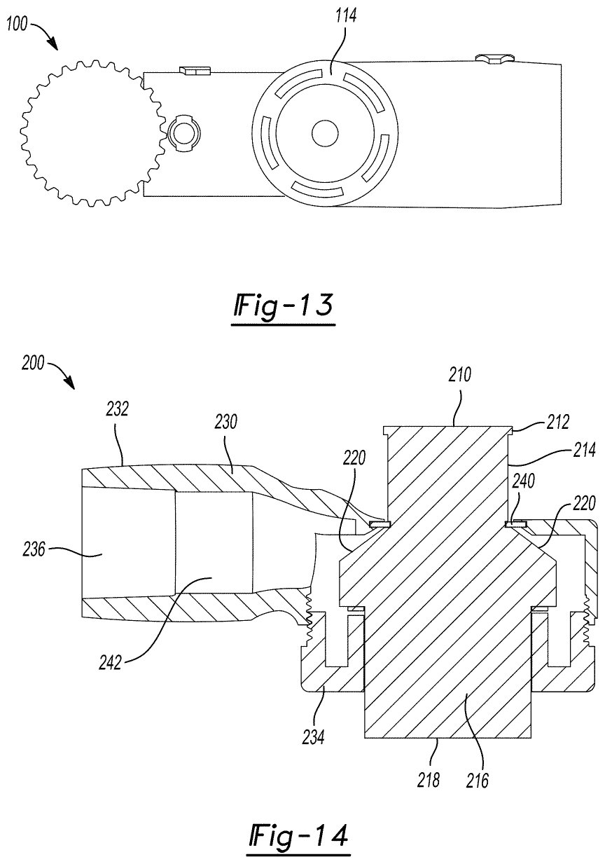

In certain aspects, the first pressure regulator may be a peak inspiratory pressure (PIP) regulator that comprises a first chamber and a second chamber, such as that generally shown in FIGS. 6-13. By way of example, FIGS. 9-10 show two views of such a first pressure regulator 100 having a first chamber 102 and a second chamber 104 in fluid communication with one another. The first pressure regulator 100 has a regulator inlet 110 in fluid communication with the first chamber 102. A regulator outlet 112 is in fluid communication with the second chamber 104. The first pressure regulator 100 also has a top portion (or body top) 114 with a centrally disposed bore or opening 116 and a threaded exterior 118.

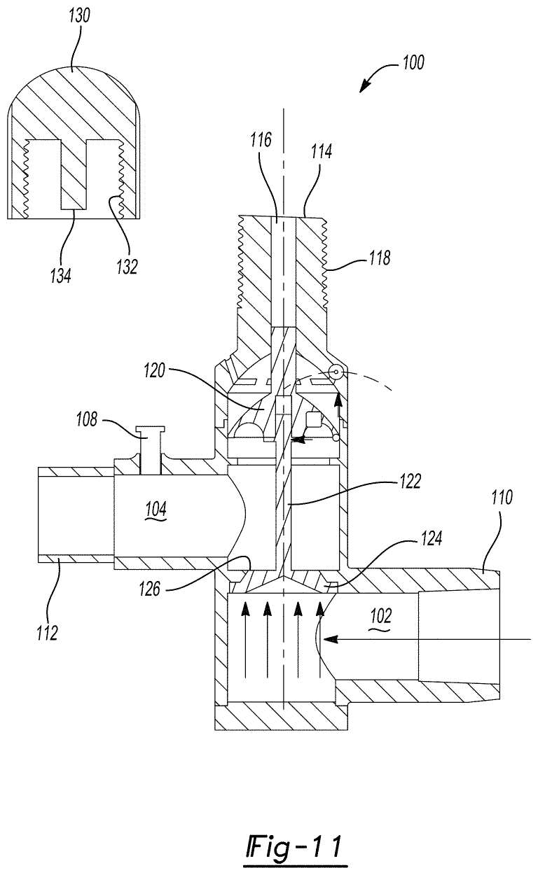

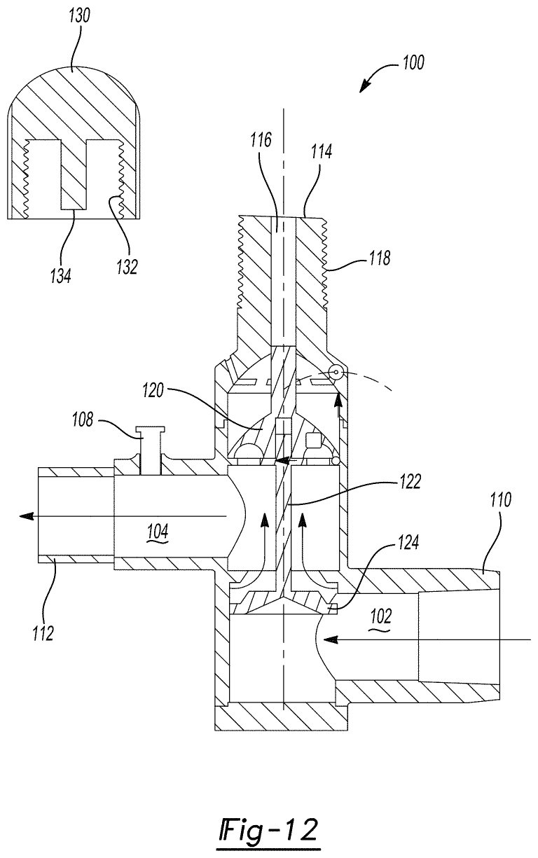

A piston 120 is disposed below the top portion 114 within the second chamber 104. Notably, the piston 120 may partially traverse into the top portion 114, as well within the second chamber 104. In addition to the piston 120, the first pressure regulator 100 may also be considered to employ a diaphragm. Thus, a seal component or plunger 122 forms a solid body that has a seat region 124 that seals against a seat 126 between the first chamber 102 and the second chamber 104. While not shown, the seat region 124 may have a gasket to seal with the seat area 126. The seal component 122 is fastened or mechanically coupled to the piston 120. In this manner, the piston 120 and the seal component 122 translate from a first operational position where fluid flow is permitted between the first chamber 102 and the second chamber 104 (see FIG. 12) to a second operational position where the seal component 122 seals the first chamber 102 from the second chamber 104 by engaging with the seat 126 to prevent fluid flow therebetween (FIG. 11).

The top portion 114 can be coupled to an adjustable cap 130. The adjustable cap has internal mating threads 132 so that it may be screwed onto the threaded exterior 118 of the top portion 114. The adjustable cap 130 also includes a centrally disposed post 134. The top portion 114 also includes a spring 140 disposed with the centrally disposed centrally disposed bore. As the adjustable cap 130 is screwed onto the threaded exterior 118 of the top portion 114, it increases compression on the spring 140 (best shown in FIGS. 9 and 10).

This allows the pressure of fluid/gas exiting the first pressure regulator 100 to be reduced with variable manual control into the output. This allows, in turn, one patient to see reduced pressure compared to a second patient. By tightening the cap 130, a healthcare professional can place more force on the spring 140 inside and thereby adjust the amount of pressure reduction performed by the first pressure regulator 100. While not shown, markers may be included for the healthcare provider to show corresponding reduction of pressure for the depth at which the adjustable cap 130 is threaded onto the top portion 114. Hence, the further the adjustable cap 130 is screwed onto the top portion 114, the more compression is applied to the spring 140 through the post 134. The spring 140 is adjacent to the piston 120 and applies force to the piston 120. The piston 120 is also acted upon via pressure in the second chamber 104 in a counter direction. While not necessary, the lower surface of the piston 120 may have features that increase a surface area.

The adjustable cap 130 thus compresses the spring 140 and places force on the piston 120. The piston 120 ultimately elevates against the spring's force when a level of pressure in the upper second chamber 104 overcomes the force of the spring 140. As the piston 120 elevates within the second chamber 104, it is attached to a separate valve system or the seal component 122 that seals the lower first chamber 102, preventing any further flow into or pressure buildup in the second chamber 104. During operation, the first pressure regulator 100 alternates between the first operational state and the second operational state. Thus, in the second operational state shown in FIG. 11 for example, a maximum pressure in the second chamber 104 is reached and the first chamber 102 is sealed off, permitting the gas to flow only out through the regulator outlet. In the first operational state as shown in FIG. 12, as the pressure reduces in the second chamber 104 as it is evacuated, it reduces the tension on the piston 120 and it reopens the first chamber 102 to the second chamber 104 by lifting the seal component 122 from the seat 126 to permit fluid to flow therebetween. Overall, the first pressure regulator 100 allows the input pressure to be reduced to a selected patient or patients with variable manual control by the healthcare provider, as described above in the context of FIG. 1.

As shown, for example in FIGS. 6-13, the first pressure regulator may also include an aperture 108 along the second chamber 104. The aperture 108 may be a standard sized IV line port, which may be capped with a universal IV line cap. Such cap is removable and the aperture 108 is connectable to any device that may be coupled to an IV line port. For example, the aperture 104 may be coupled to a pressure monitoring device, such as an arterial line pressure transducer, allowing for monitoring of the pressure exiting the first pressure regulator (e.g., allowing for controlled monitoring of the reduced pressure, P₂, being delivered to the first patient). The aperture 104 may be coupled to an oxygen line, for example, to deliver more concentrated oxygen to a single patient.

In one further aspect, a second pressure regulator may be included in the system and may be a peak inspiratory positive end-expiratory pressure (PEEP) regulator, where PEEP is the amount of pressure left in the lungs at the end of a breath. As discussed above, for the PEEP regulator, it may be advantageous to include a PEEP regulator in the system to ensure the lungs of a patient do not collapse too extensively. It is often desirable when patients are mechanically ventilated to ensure a residual pressure (e.g., 0.008 atmospheres (or about 8 cm of water), 0.0098 atmospheres (or about 10 cm of water), 0.015 atmospheres (or about 16 cm of water), etc.) remains. As shown in FIG. 1, the PEEP regulator (second pressure regulator 74) may be placed in the expiratory line between the patient and the second branched adapter.

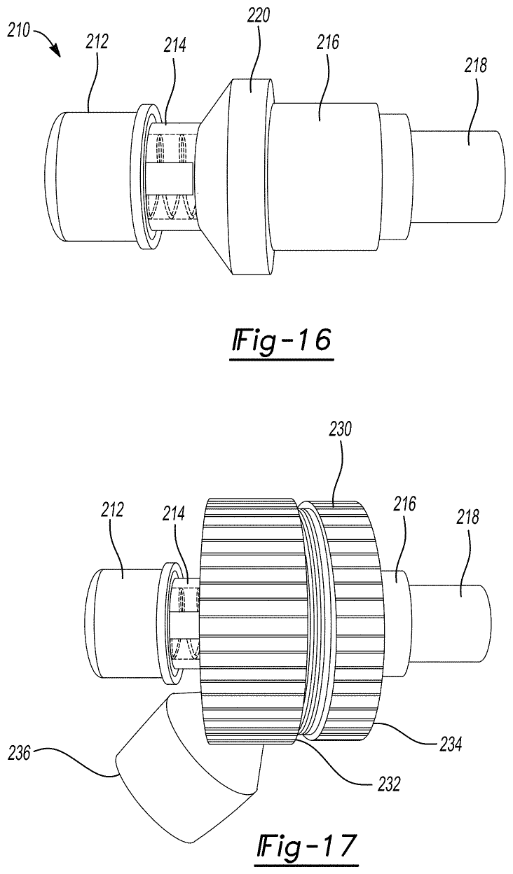

One variation of such a second pressure regulator 200 is shown in FIGS. 14-17. The second pressure regulator 200 includes a flow regulator component 210 (shown in FIG. 16, as well). As can be seen, the flow regulator component 210 has an adjustable screw top 212 threaded onto an exterior of a top portion 114, where it applies force to a fluid chamber 216. The predetermined pressure for the flow regulator component 210 may thus be set by adjusting the adjustable screw top 212. The pressure of the fluid within the line is maintained at a minimum pressure and when the pressure of the fluid entering the flow regulator component 210 exceeds a set point, the fluid is vented. Thus, a range of predetermined pressures may be set for the operation of the second pressure regulator 200.

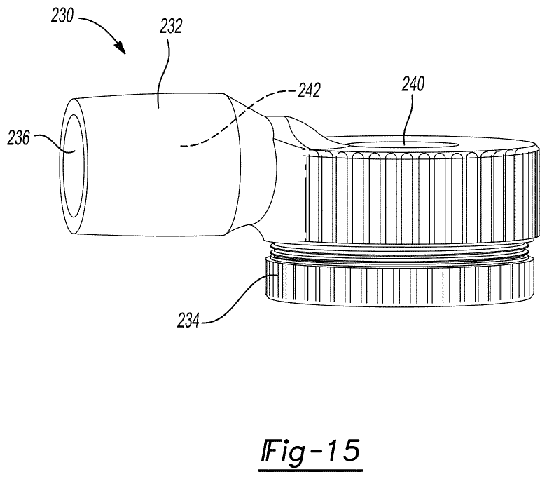

The fluid chamber 216 has an inlet 218 and an outlet that may optionally be in the form of a plurality of vents 220 through which the fluid is vented and released. A two-part housing component 230 best shown in FIG. 15, includes a first portion 232 and a second portion 234 that may be fastened together, for example, by mating threads so that the second portion 234 screws onto the first portion 232. The flow regulator component 210 may be disposed in a central opening 240 defined by the first component 232 and the second component 234. As can be seen, the plurality of vents 220 are fully enclosed within the housing 230 and thus any fluid exiting the vents 220 is directed through a fluid flow path 242 to an outlet 236. This outlet 236 may be in fluid communication with an expiratory port of a ventilator. Further, there may be one or more flow rate detectors to ascertain a volume of expiratory fluid flow that enters the ventilator. An assembly of the flow regulator component 210 and the housing 230 including the first component 232 and the second component 234 is shown in FIG. 17. As noted previously, the second pressure regulator 200 provides the advantage of capturing and thus providing the capability of measuring the expiratory fluid flow stream from a patient.

Various embodiments of the inventive technology can be further understood by the specific example contained herein. Specific Examples are provided for illustrative purposes of how to make and use the compositions, devices, and methods according to the present teachings, by are not limiting.

### Example

A multi-patient mechanical ventilation system prepared in accordance with certain aspects of the present disclosure is developed in this example to allow individualized peak inspiratory pressure settings and PEEP by using a pressure regulatory valve and an inline PEEP “booster” component. One-way valves, filters, monitoring ports and wye splitters are assembled in-line to complete the system like that shown in FIG. 1. In the following example, the system is investigated in mechanical and animal trials (ultimately with a pig and sheep concurrently ventilated from the same ventilator). The multi-patient mechanical ventilation system demonstrates the ability to provide ventilation across clinically relevant scenarios including circuit occlusion, unmatched physiology, and a surgical procedure, while allowing significantly different pressures to be safely delivered to each animal for individualized support.

In this example, the final system is manufactured and assembled in an ISO 13485 medical facility (Autocam Medical, Grand Rapids, Mich., USA) under clean conditions. Medical grade adhesive is used to secure connectors and valves to the wye pieces, and one side of the splitter is capped to allow rapid deployment in stand-by mode. Quality control testing is performed on each manufactured regulator and PEEP booster to ensure intended performance. Internal components are machined from medical-grade Radell (polyphenylsulfone) and the housing is machined from aluminum, with a silicone (platinum-cured polydimethyl siloxane) gasket seal and stainless-steel spring. Each regulator is sanitized with ethanol sonication prior to final assembly.

Initial deployment in “stand-by mode” is to a stable patient on a ventilator. At any later point, another patient can then be rapidly connected to the attachment sites for the system to use the same ventilator.

Multi-Patient Mechanical Ventilation System and Component Design

Design of components for this example are discussed herein.

Inspiratory Pressure Regulator

The inspiratory pressure regulator has components specifically designed to function across the physiologic range of expected ventilation pressures. A two-chamber system is utilized where the upper chamber is sealed from the lower chamber by a moving piston when the target pressure is reached. Once sealed, airflow into the upper chamber is halted and pressure in the inspiratory limb remains stable. The pressure at which the chambers seal can be variably adjusted by modifying the spring compression via a screw adjusted cap. The initial prototypes for the regulator were manufactured with 3D printing (Form2, FormLabs, Somerville, Mass., USA), and subsequently transitioned to a machined medical-grade aluminum product manufactured in a GMP, ISO compliant medical machining facility (AutoCam Medical). This system is found to offer substantive advantages over volume-limited or flow-limited systems. The device contains a silicone gasket, optionally fabricated out of biocompatible medical-grade, platinum-cured polydimethyl siloxane.

PEEP Booster

To regulate PEEP, an inline ball valve is manufactured with permission from Boehringer Laboratories, PEEP Valve Kit, Phoenixville, Pa. These PEEP boosters are placed in-line on the circuit in the multi-patient mechanical ventilation system. The PEEP Booster is a ball-valve system that utilizes the weight of a ⅝ inch ball in a tapered chamber to provide a constant pressure gradient across variable flow. Balls of various specific gravity (Nylon, Teflon, Stainless Steel) allow for a 2, 4, and 8 cm H₂O pressure gradient on testing.

Pressure Monitoring

A simple, widely accessible patient-specific monitoring system is incorporated into the multi-patient mechanical ventilation system circuit. In this example, an arterial line pressure transducer connected to a vital monitor follows the ventilatory pressure of each patient in real-time. The pressure loops are displayed on the patient's vital monitor, and a simple conversion from mmHg to cm H₂O can be performed by multiplying the mmHg value by 1.36. The transducer can be connected (dry) to a standard luer lock port anywhere in the circuit between the patient and the regulators and provides real-time feedback on the individual pressure loops for each patient.

Multi-Patient Mechanical Ventilation System Assembly

Inspiratory and expiratory components are identified to complete the system. Branched adapters attach directly to the circuit. A multi-patient mechanical ventilation system is developed to allow for partial predeployment on a first stable patient. In this configuration, the first patient can be maintained indefinitely without clinically meaningful changes to flow. Intravenous tubing is attached to side ports of the adapters to allow for pressure monitoring through pressure transducers. These ports are part of the assembly and can be used clinically. The wye splitters are connected to directionally specific one-way valves and the desired pressure regulator system (inspiratory pressure regulator or PEEP boosting system respectively) and are preassembled as separately packaged inspiratory and expiratory units. Preassembling the units with one-way valves decreases the assembly time for deployment and reduces risk of incorrect assembly.

In-Vitro Testing

Preliminary testing with the multi-patient mechanical ventilation system described above is first performed on linear lung simulation balloons. Leak testing of the circuit is performed, with the multi-patient mechanical ventilation system connected to an anesthesia gas machine (GE Datex-Ohmeda Aisys Carestation, General Electric, Boston, Mass., USA). A leak test is first performed using an inspiratory pressure of 50 cm H₂O and PEEP of 10, with Rate of 10/min and I:E ratio of 1:2 with flow of 15 L/m. Next, a full inspiratory hold at 60 cm H₂O is performed. Various pressure control settings are tested to validate performance. Next, a robust testing sequence is subsequently performed on a Puritan Bennet 840 ICU Ventilator [PB840] (Medtronic, Minneapolis, Minn., USA). Pressure control mode is used with a 1L test balloon on circuit 1 and a 3L test balloon on circuit 2 to test unmatched patients. A full range of physiologic pressures are tested.

Cycle Testing

Repetitive cycle testing is then performed such that a regulator is connected to a modified ventilator circuit with rapid cycling to test durability of the regulator. The system is set for the regulator to downregulate pressure to 12 cm H₂O while the ventilator drove inspiratory pressures at 25 cm H₂O. The regulator is then rapid-cycled at 96 breaths per minute.

In-Vivo Testing

The multi-patient mechanical ventilation system is further tested in animal tests using porcine and ovine models, to determine whether independent, lung protective ventilation could be delivered to two patients connected to one ventilator. All animal studies are carried out in strict compliance with the Guide for the Care and Use of Laboratory Animals of the National Institutes of Health. The protocol is reviewed by the University of Michigan University Committee on Use and Care of Animals (UCUCA) for the single pig feasibility study, and the Charles River Animal Use Committee for the combined pig and sheep study. Both studies are approved by the respective animal use committees.

Single Animal Test

First, a standard porcine model is chosen for the single animal test given respiratory physiology similar to human physiology and prior use modeling respiratory changes. Specifically, a healthy female swine weighing 71 kg is sedated with intramuscular mix of 5 mg/kg Tiletamine HCl and Zolepam HCl and 3 mg/kg Xylazine and subsequently intubated with an appropriately sized cuffed endotracheal tube and mechanically ventilated (MV) with 47% FiO₂. Total W anesthesia (TIVA) is maintained with a propofol infusion. Ventilator settings are adjusted to maintain peak inspiratory pressures <20 cm H₂O to the swine and CO₂ target between 35-45 mmHg. A catheter is placed via the internal jugular vein for administration of fluids and monitoring of central venous pressure. The multi-patient mechanical ventilation system circuit is connected to the porcine model with a 3L linear lung simulator on the other circuit and a limited volume ventilator. A series of clinical stressors and functional tests are performed with the multi-patient mechanical ventilation system to evaluate the safety of the device. At the end of each intervention, a 15 minute acclimation period is allowed for the animal prior to data collection.

Arterial blood gas (ABG) values are compared during the protocol to ensure adequate ventilation is maintained. The porcine model is connected to a balloon lung simulator via the splitting mechanism in the multi-patient mechanical ventilation system prepared in accordance with certain aspects of the present disclosure.

Pig-Sheep Dual Animal Testing

In order to validate the performance of the multi-patient mechanical ventilation system, a dual large animal model is tested with two animals of different physiology: a 43.5 kg female Dorsett crossbred sheep and an 86 kg male hybrid Yorkshire pig. Both animals are sedated, intubated and initially ventilated via separate veterinary anesthesia ventilators following the above protocol. Total intravenous anesthesia is administered for both animals without paralysis. A DigiVent DVX8′ large animal portable ventilator (Digicare Biomedical Technology, Boynton Beach, Fla., USA) is selected as the primary ventilator for the split ventilation, due to its capacity for pressure control ventilation. The swine is initially placed on the Digivent ventilator in standard pressure control ventilation in “stand-by” mode with the multi-patient mechanical ventilation system connected, but the regulated circuit capped, allowing normal ventilation to just one patient. The sheep is then connected to the pig's ventilator concurrently via the multi-patient mechanical ventilation system. Minimal adjustments are made to the ventilator to accommodate the increased flow and minute ventilation.

The animals are then subjected to a variety of physiologic scenarios, including 1) matched ventilation; 2) individualized ventilation (4 cm PEEP boost and increased PIP to the swine, baseline pressures to the sheep via inspiratory regulator); circuit occlusion; 4) physiologic stressor to one animal (superficial flap surgery performed on the swine); and 5) maximal pressure differential (increased PIP and PEEP of swine until cardiac instability is noted, while maintaining stable pressures in the sheep). Data is collected automatically through SurgiVet Data Logger System (Smith's Medical, Minneapolis, Minn., USA) at 30 second intervals.

Initial prototyping revealed that a pressure controlled system is more reliable and safe than a flow restriction technique, because circuit occlusion on one side could result in significant barotrauma to the second patient under volume control. Further prototyping confirmed a true pressure regulator is desirable for reliable performance. Large volume leaks (e.g., popoff valves or pressure relief valves) result in ventilator alarms, inadequate flow in certain ventilators, and concerns regarding aerosolized viral particles. One-way valves are used to maintain any desired pressure differential between circuits and prevent pressure equilibration, and serve a secondary function of limiting potential cross contamination. Placement of one-way valves in reverse orientation is a potential problem, and prompted the pre-assembly of a fully functional system. By sealing the system, disconnects and incorrect placement can be avoided. Placement of labels and flow direction arrows simplifies the system in a clinical setting.

Weight of the combined system is 1.2 kg and size is 27 cm×23 cm×9 cm. Because each circuit is connected to the endotracheal tube in a similar fashion to standard ventilation, a closed-line suction system can be used in each patient to limit aerosolization.

System Deployment

With the preassembled multi-patient mechanical ventilation system, deployment requires less than 1 minute to connect the system in stand-by mode for a respiratory therapist unfamiliar after a training lasting 8 minutes with the device, and less than 30 seconds to add the second patient on simulated testing.

In dual animal testing, placement of large animals into stand-by mode or dual ventilation mode took 25 seconds and 12 seconds respectively.

Pressure Monitoring

The multi-patient mechanical ventilation system incorporates a simple, widely accessible patient-specific monitoring system. A conventional arterial line pressure transducer connected to a conventional vital monitor follows the ventilatory pressure of each patient in real-time. The pressure loops are displayed on the patient's vital monitor, and a simple conversion from mmHg to cmH₂O can be performed by multiplying the mmHg value by 1.36. The transducer can be connected (dry) to a standard luer lock port anywhere in the circuit between the patient and the regulators and provides real-time feedback on the individual pressure loops for each patient.

In-Vitro Testing

Leak testing on the anesthesia gas machine demonstrates a negligible leak of less than 100 mL for all breaths at prescribed ventilation parameters, with no visible change in the plateau pressure over 2 seconds of inspiratory hold.

Initial performance testing is completed with identical simulation lungs attached to each circuit, with multi-patient mechanical ventilation system connected to the anesthesia machine. The machine is set to pressure control ventilation a peak inspiratory pressure of 36 cm H₂O while the pressure regulator is variably dialed to a range from 12 cmH₂O up to 36 cmH₂O. The PEEP is initially set to 5 cm H₂O on the anesthesia machine, and PEEP boosters are then added to the circuit, confirming a fully individualized pressure control could be maintained on each circuit. For any desired ventilation pressures, the ventilator is set to the highest planned Peak Inspiratory Pressure and the lowest planned PEEP.

Similarly, a robust benchtop testing performed on the PB840 tested ventilation pressures ranging from 15/5 to 45/20 (PIP/PEEP) confirms the performance of the multi-patient mechanical ventilation system prepared in accordance with certain aspects of the present disclosure. Regulated pressures are tested from 60% to 100% of the PIP at all testing conditions with success, and the 2, 4, and 8 cm H₂O PEEP boosters all functioned as intended, allowing a boost in PEEP from 2-14 cm H₂O above baseline (boosters can be “stacked” as needed). Various I:E ratios are tested ranging from 1:1-1:5 as well as pressure slope from 5-100%, all of which demonstrated reliable performance in the regulator across the full spectrum of pressure control ventilation.

Cycle Testing

The rapid-cycle testing completed 600,000 continuous cycles on one of the multi-patient mechanical ventilation system regulators over 5 days at 96 cycles/minute. Ventilator pressure remained stable throughout the test at 25 cm H₂O and the regulator maintained a downregulated pressure of 12 cmH₂O stably throughout the test. The regulator is subsequently disassembled, with no evidence of wear or degradation on the system.

In-Vivo Testing

Single Animal Test

The study is conducted over a five-hour total duration. The results demonstrate that the pig could be safely ventilated at stable pressures while varying the pressures delivered to the balloon across a wide range. The study also confirmed that a standard arterial line pressure transducer provides excellent real-time monitoring of ventilation pressures, and although the ventilator is limited to volume-controlled ventilation modes, it is run based off pressure readings analogous to pressure control ventilation. Simulated coughing and dyssynchrony in the balloon (manual squeezing) did not result in significant ventilatory changes to the swine due to the function of the 1-way valves. In open and occluded circuit scenarios, the swine is protected from barotrauma, but did demonstrate signs of hypoventilation that are immediately alarmed on the ventilator and monitors. The study demonstrated the pressure regulator could safely control the inspiratory pressure to the swine from 12-17 cm H₂O while the inspiratory pressure in the balloon is increased up to 22 cm H₂O, while maintaining stable ventilation for the swine. Arterial blood gas monitoring confirmed the pig remained well ventilated with stable parameters even when inspiratory pressures are regulated by the pressure regulator. The PEEP boosters provide reliable increases in PEEP on the applied circuit. Given that the system is entirely housed at the ventilator, movement of the swine and balloon result in no changes to the performance.

Disparate Dual Animal Test

The dual animal study is completed over a 6 hour duration. The small DigiVent ventilator is a portable ventilator that is readily capable of generating adequate volume and flow to support two large animals from the single ventilator, and is readily assembled as an ICU style ventilator. There are no complications during the testing. Connecting the swine into stand-by mode required 25 seconds, while connecting the sheep to the second circuit required 12 seconds. Small adjustments are made to the ventilator settings after split ventilation is established to accommodate for the increased flow and volume requirements of the ventilator (increased respiratory rate by 1 BPM). Several scenarios are tested for at least 15 minutes duration, including matched ventilation, various increased pressures for the swine, and a superficial surgical flap dissection in the swine to simulate physiologic stress. The flap procedure comprises elevating and exposing the abdominal skin and soft tissues with electrocautery, and lasts 40 minutes. During the procedure, the animals remain very stable with no need for adjustments. Comparison of the ventilatory data demonstrates both animals could be safely coventilated at different pressures for prolonged periods.

Statistical analysis of the composite data from the data logger demonstrates that the swine and sheep ventilation pressures are statistically different once regulated in both PIP and PEEP and yet, there was no difference in the sheep's ventilation pressures throughout all testing parameters. Arterial blood gas analysis throughout the experiment correlated with end-tidal CO₂ readings and SpO₂ and confirms the animals are maintaining adequate ventilation. Similar to the single-animal experiment, one circuit disconnect resulted in significant hypoventilation of both animals, however circuit occlusion resulted in no significant changes for the second animal, especially with pressure control ventilation. The largest ventilation pressure differential tested was 12 cmH₂O between the swine and sheep (PIP/PEEP 33.4/13.7 vs. 21.3/1.4 respectively). In this scenario, both animals are being ventilated, but the high pressures ultimately caused cardiac arrhythmias in the swine and the experiment was terminated.

The multi-patient mechanical ventilation system has been efficacious in mechanical simulations and animal experiments, providing individualized pressure-control ventilation and monitoring. Accordingly, a compact multi-patient mechanical ventilation system (capable of enabling mechanical ventilation for multiple patients from a single ventilator) potentially addresses many problems associated with acute shortages of ventilators in clinically important settings, including the current COVID-19 pandemic. This led to an initial development of split ventilation used in pair-matched human use, including use on patients during the COVID-19 pandemic. The present disclosure provides a system that can be developed to enable expanded access to life saving ventilatory support in a pressure control ventilation mode (rather than being limited to volume control), while addressing concerns related to simple “ventilator splitting.” The multi-patient mechanical ventilation system differs from all previous work with a new pressure regulator component custom designed for ventilator pressures, and manufactured in an ISO compliant facility. This combined with PEEP boosters are not volume restricting and allow for differing pressures to be applied.

It has been demonstrated that it is possible to individualize, protect, adapt, and control ventilation in a complex, disparate dual-animal model for over 6 hours with multiple interventions. Further, it is demonstrated that one patient can experience a wide breadth of physiologic stressors and ventilatory changes, while the second patient can remain stable with unchanged parameters. This specifically addresses concerns for over/under ventilation of patients with different lung compliances. The standby mode and the rapidity with which patient can be placed on or removed from a joint circuit are especially advantageous.

The multi-patient mechanical ventilation system provided in accordance with certain aspects of the present disclosure addresses most major concerns raised regarding ventilator splitting, for example, managing differential compliance and PEEP requirements, personalized monitoring with alarm capacity, a disconnected circuit can be simply capped if needed, and circuit occlusion does not significantly affect ventilation to the second patient.

Moreover, the multi-patient mechanical ventilation system are available at a fraction of the cost, footprint and weight that would be required for comparably capable, full size ventilators. Thus, they can be deployed more rapidly than ventilators, which may make rapid and agile delivery to remote or lower-resourced locations more facile. A system can be setup and delivered in much less time than that for a full ventilator. The utilization of a standard arterial line pressure transducer and monitor allows the ability to individually monitor each patient's ventilation pressures in real time remotely. In certain aspects, the systems may be preassembled to reduce potential errors in setup. In settings of limited ventilator availability, delivery systems can be developed to allow increased delivery of ventilator support to enable rapid deployment under constraints of time, space and finances. It is also contemplated that such ventilation systems could be further engineered to allow different pressures to be delivered between lungs of a single individual.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for providing mechanical ventilation to a plurality of patients using a ventilator, the system comprising:
a first branched adapter comprising a first inlet and a plurality of first outlets fluidly coupled with the first inlet, wherein the first inlet is in fluid communication with an inspiratory port of the ventilator to receive inspiratory fluid therefrom and each of the plurality of first outlets are in fluid communication with a plurality of conduits to each of the plurality of patients and configured to provide inspiratory fluid thereto;
a second branched adapter comprising a plurality of second inlets and a second outlet fluidly coupled with the plurality of second inlets, wherein each of the plurality of second inlets are in fluid communication with a plurality of conduits from each of the plurality of patients and configured to receive expiratory fluid flow therefrom and the second outlet is in fluid communication with an expiratory port of the ventilator; and
a first pressure regulator comprising a regulator inlet that is in fluid communication with at least one of the plurality of first outlets of the first branched adapter and a regulator outlet fluidly coupled to the regulator inlet and in fluid communication with at least one of the plurality of patients, wherein the first pressure regulator is configured to receive the inspiratory fluid via the regulator inlet and reduce a pressure of the inspiratory fluid that exits the regulator outlet.

2. The system of claim 1, wherein the first inlet of the first branched adapter is directly or indirectly connectable to the inspiratory port of the ventilator, the second outlet of the second branched adapter is directly or indirectly connectable to the expiratory port of the ventilator, and the regulator inlet of the first pressure regulator is directly or indirectly coupled to one of the plurality of first outlets of the first branched adapter.

3. The system of claim 1, wherein the first pressure regulator is manually tunable to control an amount of pressure reduction performed.

4. The system of claim 1, wherein the first pressure regulator comprises: a first chamber fluidly coupled to the regulator inlet, a second chamber fluidly coupled to the regulator outlet and the first chamber, a second outlet in fluid communication with the second chamber, an adjustable cap disposed over the second chamber, wherein the adjustable cap is configured to manually adjust a pressure of fluid exiting the second outlet.

5. The system of claim 1, wherein the first pressure regulator comprises: a first chamber fluidly coupled to the regulator inlet, a second chamber fluidly coupled to the regulator outlet and the first chamber, a second outlet in fluid communication with the second chamber, an adjustable cap disposed over the second chamber, a spring disposed beneath the adjustable cap, a piston at least partially disposed within the second chamber, wherein the spring is configured to apply compressive force to the piston via the adjustable cap, and a seal component coupled to the piston, wherein the piston and the seal component translate from a first operational position of the first pressure regulator where fluid flow is permitted between the first chamber and the second chamber to a second operational position where the seal component seals the first chamber from the second chamber to prevent fluid flow.

6. The system of claim 1, wherein at least one of the first pressure regulator, the first branched adapter, and the second branched adapter has a port for fluid communication with a pressure monitor.

7. The system of claim 1, further comprising a second pressure regulator, the second pressure regulator comprising a flow regulator component comprising an inlet and an outlet, and a housing encasing at least a portion of the flow regulator component and configured to receive fluid exiting the outlet of the flow regulator component and direct it to an outlet of the housing.

8. The system of claim 7, further comprising circuit tubing connectable to the first branched adapter, the first pressure regulator, the second pressure regulator, the second branched adapter, and a patient-interfacing device.

9. The system of claim 8, wherein the circuit tubing comprises a tube and forms a first conduit through which an inspiratory gas stream can flow from the first branched adapter, through the first pressure regulator, and through the patient-interfacing device to one of the plurality of patients or forms a second conduit through which an expiratory gas stream can flow from one of the plurality of patients, through the patient-interfacing device, optionally through the second pressure regulator, and through the second branched adapter to the ventilator.

10. The system of claim 1, further comprising a one-way valve.

11. The system of claim 10, wherein the one-way valve is disposed within a branch of the first branched adapter or second branched adapter or proximal to and in-line with the first branched adapter or the second branched adapter.

12. An adapter assembly for a ventilator system that provides mechanical ventilation to a plurality of patients using a ventilator, the adapter assembly comprising:
a first branched adapter comprising a first inlet and a plurality of first outlets fluidly coupled with the first inlet, wherein the first branched adapter is configured to be connected to conduits to the plurality of patients and configured to receive inspiratory fluid from the ventilator;
a second branched adapter comprising a plurality of second inlets and a second outlet fluidly coupled with the plurality of second inlets, wherein the second branched adapter is configured to be connected to conduits from the plurality of patients and configured to receive expiratory fluid flow from the plurality of patients; and
at least one pressure regulator comprising a regulator inlet that is in fluid communication with a regulator outlet fluidly coupled to the regulator inlet, wherein the at least one pressure regulator adjusts or maintains a pressure of at least one of the conduits.

13. A method of providing mechanical ventilation to a plurality of patients using a single ventilator, the method comprising:
attaching a first branched adapter directly or indirectly to an inspiratory port of a ventilator, the first branched adapter having a plurality of branches and adapted to divide an inspiratory fluid released by the ventilator at a first pressure into a plurality of inspiratory streams for delivery to a plurality of patients;
attaching a second branched adapter directly or indirectly to an expiratory port of the ventilator, the second branched adapter having a plurality of branches and adapted to unite a plurality of expiratory streams emitted by the plurality of patients into a single flow of expiratory fluid for return to the expiratory port;
attaching a pressure regulator directly or indirectly to one of the plurality of branches of the first branched adapter, wherein the pressure regulator is configured to receive a first of the plurality of inspiratory streams at the first pressure and reduce the first pressure to a second pressure; and
attaching a plurality of conduits to fluidly couple each of the plurality of patients to a respective branch of the first branched adapter and a respective branch of the second branched adapter such that each patient is connected to a respective inspiration line and a respective expiration line, wherein a first patient is connected to a first inspiration line having the pressure regulator disposed along and in fluid communication with the first inspiration line; wherein during operation, the ventilator is configured to release the inspiratory fluid at the first pressure and the first patient receives a stream of fluid at the second pressure.
